(19) United States
(12) Patent Application Publication
(10) Pub. No.: US 2007/0178057 A1
(43) Pub. Date: Aug. 2, 2007

(54) High efficiency sunscreen composition particularly useful for wipes and sprays

(75) Inventors: Ashoke K. SenGupta, Barrington, IL (US); Kevin Cureton, Evanston, IL (US); Ilona Lin, Wauconda, IL (US); Thomas W. Beihoffer, Arlington Heights, IL (US)

Correspondence Address: MARSHALL, GERSTEIN & BORUN LLP, 233 S. WACKER DRIVE, SUITE 6300, SEARS TOWER, CHICAGO, IL, 60606, US

(73) Assignee: AMCOL International Corporation, Arlington Heights, IL (US)

(21) Appl. No.: 11/345,064

(22) Filed: Feb. 1, 2006

(51) Int. Cl. A61K 8/00; A61K 8/18; A61Q 17/04

(52) U.S. Cl. 424/59

(57) ABSTRACT

A sunscreen composition, either impregnating a wipe substrate or comprising a sunscreen spray, wherein the composition comprises an oil phase dispersed stably as emulsion droplets in a water phase that contains i) a booster for the sun protection factor (SPF), comprising a combination of water-dispersible particulate materials, one of which is smectite clay, and a water-soluble or water-dispersible phenolic polymer; and ii) a water-soluble or water-dispersible polymer having a weak acid group, a weight average molecular weight of 1,000-100,000 Dalton, and an anionic charge density of no less than 4 milliequivalent per gram of the polymer; the said sunscreen composition meeting the following specifications: i) the in-vivo sun protection factor (SPF) is ≧1.8 times the weight percent of the sunscreen active(s) contained therein, based on the weight of the sunscreen composition; and ii) the shear thinning index of the composition is greater by 5% or higher with the weak acid polymer contained therein than without the polymer, the shear thinning index being defined as the ratio of viscosities of the sunscreen composition at 0.5 rpm and 20 rpm, measured on a Brookfield RVT viscometer at 25 ° C., using spindle 7, after cooling the composition to room temperature and storing it at that temperature for about 24 hours.

### FIELD OF THE INVENTION

The present invention relates to compositions for sunscreen wipes and sprays, which allow an efficient transfer of sunscreen actives to the skin. In the preferred embodiments, it relates to high-efficiency (i.e., with enhanced sun protection factor) sunscreen compositions for sun protection wipes and sprays, which enable sunscreen actives contained therein to be transferred to the skin efficiently, due to having certain desirable rheological properties, and stability. The preferred composition is in the form of a highly shear-thinning, stable, oil-in-water (O-W) emulsion, with at least one water-insoluble, organic sunscreen active contained in the oil phase, while the water phase contains i) an SPF (sun protection factor) booster, comprising a mixture of particulate materials including smectite clay, and an interfacially-active phenolic polymer; and ii) a low molecular weight, non-thickening, water-soluble polymer, preferably selected from alkali metal salts of weak acid polymers, wherein the sunscreen emulsion exhibits an increased shear-thinning due to having the weak acid polymer as a component.

### BACKGROUND OF THE INVENTION

It is well recognized that the solar ultraviolet (UV) radiation poses serious threat of human skin damage which may range from the short term hazard like erythema, i.e., sunburn, to long term hazards like skin cancer and/or premature aging of the skin. The SPF rating system has been developed to help consumers select the appropriate sun protection product for any given outdoor activity involving exposure to the sun. The SPF number corresponds to a multiplying factor by which the duration of protection by a properly applied sunscreen exceeds the exposure time that causes the unprotected skin to show darkening. Thus, with proper application of an SPF 15 product, a person should be able to remain in the sun without skin darkening for fifteen times the usual unprotected duration. In recent years, due to the increased public awareness of UV radiation hazards, the use of sun protection products has grown considerably. Among these products, wipes and sprays are gaining increasing consumer preference because of the convenience in product application.

Most skin care products in the form of creams and lotions are essentially emulsions, having either an oil phase emulsified in a water phase (O-W emulsions) or a water phase emulsified in an oil phase (W-O emulsions). Typically, a thickened continuous phase (e.g., the water phase in O-W emulsions) tends to enhance emulsion stability against phase separation induced by flocculation, Ostwald ripening, sedimentation (creaming) as well as coalescence of emulsion droplets. On the other hand, viscous compositions, generally, fall short of being adequately delivered to the skin from wipes and sprays. Fulfilling the conflicting demands of having emulsion compositions that transfer easily from wipes onto the skin, yet, while remaining stable, is a challenge that is met in accordance with the present invention.

For wipes having a basesheet or fiber-containing substrate impregnated with an unstable emulsion, as the emulsion droplets grow bigger in size due to coalescence, an effect that eventually leads to the separation of the emulsified phase, the large droplets thus formed tend to deposit onto the wipe substrate due to various phenomena including the following: i) increased van der Waals attraction between emulsion droplets and the wipe substrate with increasing particle size; and ii) mechanical interception of large emulsion droplets by the fibers of the basesheet. Such deposition of emulsion droplets onto the basesheet could greatly reduce the skin-delivery of a skin care active contained in the emulsion droplets. In O-W sunscreen emulsions, the sunscreen actives are generally incorporated into the emulsified oil phase, being oil-soluble or oil-dispersible. Clearly, only a small fraction of the sunscreen actives would be delivered to the skin, leaving the skin virtually unprotected against UV radiation, if sunscreen wipes contained unstable O-W sunscreen emulsions. It is imperative, therefore, for O-W emulsion-based products to be highly stable, in order to ensure an efficient transfer of the emulsified sunscreen actives from wipes to any given substrate.

Most organic sunscreen agents are oil-like and/or oil-soluble materials that are often expensive. High levels of these actives in sun care products increase the cost of the products, while rendering the products less appealing for their greasy skin feel and skin irritation. One way to address these issues would be to include an ingredient that functions as an SPF booster, being capable of increasing the SPF significantly, despite not being a strong UV-absorber at its typical use level.

Polargel® UV, a sunscreen additive from AMCOL International Corporation, is a proven ingredient for boosting the SPF of sunscreen emulsions that contain water-insoluble, organic sunscreen actives (see U.S. Pat. Nos. 6,500,411 B2 and 6,716,418). It comprises a mixture of particulate materials including smectite clay, with an interfacially-active phenolic polymer, e.g., lignosulfonate, used as a dispersant or surface-modifier for the particulate materials. These particulate materials, with relatively high specific gravities, exhibit high settling rates even for particles less than 1 micron in size, in less viscous aqueous compositions.

Polargel® UV enables achieving a given SPF for an O-W sunscreen emulsion, but with a lower dosage of organic sunscreen actives contained in the emulsified oil phase than otherwise. However, its effective use in O-W sunscreen emulsions relies on having its particulate constituents remaining suspended in the water phase of the emulsions, in turn requiring the emulsions to be viscous under storage conditions. This requirement is particularly critical for sunscreen wipes since settling of the particulate constituents in the relatively thin emulsion mass impregnating a single wipe basesheet could lead to a rapid loss of these SPF-boosting constituents from the skin-contacting surface of the basesheet.

The prior art related to sunscreen wipes, for example, published US patent applications 2003/0012809 A1 and 2004/0228811 A1, describes sunscreen compositions with a low-shear-rate viscosity in the range of 0-20,000 cps. Nonetheless, low-shear-rate viscosities would have to be much greater than 20,000 cps in order to maintain good suspension of a particulate-based additive such as Polargel® UV, in the water phase of O-W emulsions. These prior art sunscreen compositions, therefore, could not possibly include a SPF booster comprising of water-dispersible particulate materials, such as Polargel® UV, while maintaining good suspension of the additive's particulate constituents in the water phase, a critical requirement for realizing the efficacy of such an SPF booster.

A common method for thickening the water phase of O-W emulsion-based personal care and cosmetic compositions is to use water-soluble, polymeric thickeners and/or particulate material-based thickeners, such as smectite clay and fumed inorganic oxides (e.g., silica). The most widely used polymeric thickeners include crosslinked acrylic acid polymers, xanthan gum, and cellulosic polymers. It is known in the art that combining any of these polymers with smectite clay results in synergy in thickening. Accordingly, with any of these polymers used as the primary thickener for an O-W emulsion, the emulsion viscosity would be considerably higher if the emulsion contains a smectite clay-laden additive such as Polargel® UV. The increased viscosity is expected to have a positive impact on emulsion stability, but it would render the emulsion unsuitable for wipe and spray products, as there would be a less efficient transfer of the emulsion onto the skin from these products, if the emulsion is more viscous but while not highly shear-thinning.

In the light of all of the above, and in particular the benefits of including a water-borne SPF booster such as Polargel® UV in a sunscreen formulation, it is an object of the present invention to produce sunscreen wipe and spray compositions comprising a highly stable, O-W emulsion-based sunscreen formulation that contains, in the water phase, a phenolic polymer, a smectite clay particulate together with one or more additional particulate materials having a primary particle size of less than 1 micron, and a low molecular weight, weak-acid polymer having a weight average molecular weight in the range of 1,000-100,000 Dalton, with the weak acid polymer enabling the sunscreen composition to exhibit certain desirable viscosity properties, specific to sunscreen wipes and spray compositions, while maintaining good suspension of the particulate constituents in the water phase of the emulsion.

### SUMMARY

Described herein are sunscreen compositions in the form of wipes and sprays, comprising an oil-in-water (O-W) sunscreen emulsion that meets certain specifications related to sun protection factor (SPF), viscosity, shear-thinning, and stability, wherein the wipes comprise a wipe substrate impregnated with the said sunscreen emulsion. More particularly, the O-W sunscreen emulsion contains, in the water phase, a water-borne SPF booster, and a low molecular weight, weak-acid polymer, while in the oil phase, at least one water-insoluble organic sunscreen active. The SPF booster additive comprises a phenolic polymer, and a mixture of water-dispersible particulate materials, one of which is smectite clay, wherein at least one of the particulate components has a primary particle size of less than 1 micron. The phenolic polymer serves as a dispersant or surface-modifier for the particulate components of the additive, and is further capable of functioning as an emulsifier for O-W emulsions.

In order to ensure that the particulate components of the SPF booster can remain suspended in the water phase of the sunscreen emulsion compositions described herein over extended storage durations, the emulsion's water phase is thickened with a thickening agent. The thickening agent is preferably selected from high molecular weight, water-soluble or water-dispersible polymers known in the art as thickening agents, and particulate-based thickening agents such as smectite clay and fumed inorganic oxide (e.g., silica). The thickening agents may further include the liquid-crystalline structure forming materials such as fatty acids, fatty esters, and fatty alcohols.

For the O-W sunscreen emulsions of the present invention, the ratio of the SPF to the total amount, expressed as % by weight of the sunscreen emulsion, of organic sunscreen active(s) contained in the emulsion is at least 1.8. In other words, the SPF of the sunscreen emulsion described herein is at least 18, if the organic sunscreen content is 10% by weight of the emulsion.

These sunscreen emulsions should have low-shear-rate viscosities of at least 50,000 cps at 0.5 rpm and 35,000 cps at 1 rpm, while the high-shear-rate viscosities are 10,000-30,000 cps at 5 rpm and 2,500-10,000 cps at 20 rpm, as measured on a Brookfield RVT viscometer, at 25° C., using spindle # 7. As a critical requirement for the sunscreen compositions described herein, the sunscreen emulsions contain a shear-thinning-boosting ingredient that is capable of increasing the shear-thinning index of the compositions by at least 5% over the shear-thinning index of the compositions without the shear-thinning-boosting ingredient (when the ingredient is replaced by the same weight of water), where shear-thinning index is a term used herein for quantifying the level of shear-thinning. As defined herein, shear-thinning index is the ratio of emulsion viscosities at 0.5 rpm and 20 rpm, with the viscosities measured on a Brookfield RVT viscometer, at 25° C., using spindle # 7. Unexpectedly, the inclusion of a low molecular weight weak acid polymer as a water-phase component for the O-W sunscreen emulsions described herein, enables achieving a 5% or greater increase in the shear-thinning index.

Furthermore, to achieve the full advantage of the sunscreen emulsions described herein, the emulsion has no visible separation of the oil phase or the particulate components of the water phase, when centrifuged at 3,000 rpm for 30 minutes after being heated to 60° C. In a preferred embodiment, the sunscreen emulsions also show good stability against any separation of the oil phase and the particulate components of the water phase, when subjected to freeze-thaw stability testing involving the following: i) freezing the emulsion sample at −10° C. for a period of 24 hours, followed by thawing the emulsion sample at room temperature; ii) repeating these steps at least three times on the same emulsion sample; and iii) repeating (i) and (ii) with sunscreen wipes made after impregnating a wipe-substrate with the said emulsion.

### DETAILED DESCRIPTION

Described herein are compositions for sunscreen wipes and sprays, comprising a sunscreen emulsion that meets certain specifications related to sun protection factor (SPF), viscosity, shear-thinning, and stability properties. In one embodiment, the sunscreen emulsion impregnates a water-insoluble wipe substrate or basesheet. By “water-insoluble” is meant the wipe substrate does not dissolve in or disintegrate upon immersion in water. Nonlimiting examples of such substrates include both nonwoven substrates and woven substrates known in the art, made from polymeric and/or natural fibers. The amount of the sunscreen emulsion relative to the weight of the wipe substrate may range from about 20:1 to about 1:1, more preferably from about 15:1 to about 10:1, and most preferably from about 6:1 to about 2:1.

The preferred sunscreen emulsion composition is produced in the form of an oil-in-water (O-W) emulsion, wherein the oil phase, as is typical in prior art sunscreen compositions, contains at least about 2% by weight of a (any) water-insoluble, organic sunscreen active, based on the weight of the emulsion.

In accordance with the O-W sunscreen-emulsion compositions described herein, it has been found that by incorporating, into the water phase, a water-borne SPF booster comprising a water-soluble or water-dispersible phenolic polymer and a combination of water-dispersible particulate materials, one of which is smectite clay, the ratio of the SPF (in-vivo SPF measured as per the standard protocols known in the art, preferably under the very water-resistant SPF testing conditions) of the sunscreen emulsion to the total amount (expressed as % by weight of the emulsion) of the sunscreen actives contained therein, can be at least as high as 1.8, provided that one of the particulate components of the SPF booster has a primary particle size of less than 1 micron. One particularly effective SPF booster is Polargel® UV containing about 0.5% to about 40% by weight of smectite clay, together with about 0.5% to about 40% of another particulate material, and about 0.5% to about 20% of a phenolic polymer. A low molecular weight weak-acid polymer is included in the water phase of the sunscreen emulsion to provide unexpected viscosity characteristics for the emulsion, that enable maintaining sedimentation-stability of the SPF-boosting particulate components contained in the emulsion's water phase, while ensuring that the emulsion can be transferred adequately to the skin from wipe and spray form.

The water phase of the sunscreen emulsions described herein should contain smectite clay in an amount of about 0.1% to about 5% by weight, preferably about 0.5% to about 2% by weight, and more preferably about 1% to about 1.5% by weight. The amount of phenolic polymer contained (dissolved or dispersed) in the water phase should be in the range of about 0.025% to about 2.5%, preferably about 0.05% to about 1% by weight, and more preferably about 0.1% to about 0.5% by weight. The additional particulate material (in addition to the smectite clay) should be included in the water phase in an amount of about 0.1% to about 20%, preferably about 0.3% to about 5% by weight, and more preferably about 0.5% to about 3% by weight. When the SPF booster is supplied from Polargel® UV, the dosage of Polargel® UV may range from about 0.5% to about 35%, more preferably from about 1% to about 20%, and most preferably from about 2% to about 15%, based on the weight of the water phase of the sunscreen emulsion in order to achieve the final water phase composition of the sunscreen emulsion, detailed above.

The preferred SPF booster comprises a mixture of water-dispersible particulate materials at least one of which is smectite clay, and a phenolic polymer, serving as dispersant/surface-modifier for the particulate materials. The particulate component that is combined with the smectite clay can be any particulate material selected from inorganic oxides, water-insoluble inorganic salts, silicate minerals, and water-insoluble organic particulate materials, whose surface can be modified by the adsorption of the phenolic polymer on the particle surface. Preferred examples of these particulate materials for combination with the smectite clay include titanium dioxide, zinc oxide, alumina, silica, talc, and latex polymers. The essential smectite clay particulate component of the SPF booster is selected from bentonite, montmorillonite, saponite, hectorite, bidelite, and/or stevensite. The ratio of the amount of smectite clay to the amount of the non-smectite clay particulate component(s) of the SPF booster is in the range of 1:1-1:30. To achieve the full advantage of the sunscreen emulsions descried herein, at least one particulate component of the SPF booster should have a primary particle size of less than 1 micron, preferably less than 0.5 micron, and most preferably less than 0.25 micron. In a preferred embodiment, the SPF booster is preferably an aqueous dispersion of the foregoing particulate components, wherein the smectite clay remains in a highly exfoliated form (i.e., wherein the clay platelets are delaminated or separated from one another across their face surfaces).

The dispersant/surface modifier for the particulate materials is either a phenolic polymer, or a mixture of a phenolic polymer and an alkali metal salt of an acrylic acid polymer. Non-limiting examples of the phenolic polymer include lignosulfonate, lignin, oxylignin, and humate.

The water phase of the sunscreen emulsion compositions described herein is thickened using a thickening agent known in the art, in order to ensure good suspension of the particulate materials of the SPF booster, e.g., Polargel® UV. The thickening agent is selected preferably from high molecular weight (with a weight average molecular weight of >500,000 Dalton) polymeric thickeners, particulate-based thickeners such as smectite clays, and mixtures thereof. The polymeric thickener is preferably an anionic polymer, and most preferably a crosslinked acrylic acid polymer, non-limiting examples of which include the following polymers listed by their respective International Nomenclature Cosmetic Ingredient name: Acrylates/C10-30 Alkyl Acrylates Crosspolymer and Carbomer. The amount of the high molecular weight polymeric thickener in the water phase of the sunscreen emulsions described herein is in the range of from about 0.05% to about 5%, preferably in the range of from about 0.1% to about 1%, and most preferably in the range of from about 0.15% to about 0.5%, based on the weight of the emulsion. The most preferred smectite clay-based thickener is sodium bentonite, comprising about 0.1-5%, preferably 0.25%-2%, and most preferably 0.5-1% by weight of the water phase of the sunscreen emulsion. With the use of the foregoing thickening agents, the sunscreen emulsions are required to have low-shear-rate viscosities of at least 50,000 cps at 0.5 rpm and 30,000 cps at 1 rpm, while the high-shear-rate viscosities are 10,000-35,000 cps at 5 rpm and 2,500-10,000 cps at 20 rpm, as measured on a Brookfield RVT viscometer, at 25° C., using spindle # 7.

The water phase of the sunscreen emulsion of the present invention further contains a water-soluble or water-dispersible shear-thinning-boosting ingredient selected, preferably, from alkali metal salts of weak acid polymers and copolymers having a weight averaged molecular weight of 1,000-100,000 Dalton and an anionic charge density of no less than 1.5 milliequivalents per gram of the polymer. Non-limiting examples of such preferred shear-thinning aids include polyacrylate, polyphosphate, polyphosphonate, polyphenolate, and mixtures thereof. The polymer is necessarily such that, when used at or above a certain threshold dosage that may vary from one polymer to another, the ratio of emulsion-viscosities (measured on a Brookfield RVT viscometer, using spindle # 7) at 0.5 rpm and 20 rpm is at least 5% greater as compared to when the emulsion does not contain the polymer, i.e., when the polymer is replaced by an equivalent amount of water.

The water phase of the sunscreen emulsion of the present invention may also contain a polymeric emulsifier for stably dispersing the oil phase within the water phase of the emulsion. This is to minimize the use of detersive surfactants as an emulsifier, since these surfactants tend to mar the water resistance property of the sunscreen emulsion, especially when used at relatively high dosages. The polymeric emulsifier is selected from water-soluble or water-dispersible amphiphilic copolymers, polyalkyl glucoside with an alkyl chain length of C8-C30, and hydrophobically-modified, water-soluble or water-dispersible polymers.

The sunscreen emulsions described herein may further contain, in either the oil phase or the water phase, emulsifiers, emollients, fatty acids, alcohols and esters, oil-phase thickening agents such as oragnoclays, waxes, and polymeric thickeners, hydrophilic liquids such as glycols and glycerin, chelating agents, waterproofing agents, film-forming agents, moisturizing agents and humectants, sensory property boosting agents, antioxidants, vitamins, preservatives, fragrances, coloring pigments and dyes, water-insoluble particulate material-based SPF boosters of inorganic and/or organic origin (for example, SunSpheres™ from Rohm and Haas Company), and surface-modified particulate materials, known in the art.

The following examples will more fully illustrate the preferred embodiments within the scope of the present invention. These examples are solely for the purpose of illustration and are not to be construed as limitations of the present invention as many variations thereof are possible without departing from the purview and spirit of the invention.

### EXAMPLE I

This example demonstrates the benefit of incorporating an SPF booster, Polargel® UV 1116, from AMCOL International Corporation, in the O-W emulsion-based sunscreen compositions described herein, inasmuch as the present composition required considerably lower amounts of sunscreen actives as compared to O-W emulsion-based sunscreen compositions in the prior art, for attaining comparable SPF values. Table I shows the sunscreen active contents of the various formulations along with their respective SPF values, while Table II shows the sunscreen composition of the present invention.

TABLE I

| Formulation | Sunscreen Actives | SPF |
|---|---|---|
| Sunscreen Composition of | 5% Octyl Methoxycinnamate, 3% | 32 |
| the Present Invention | Octyl Salicylate, 3% Oxybenzone, | |
| | 2% Avobenzone | |
| Neutrogena ® Active | 7.5% Octinoxate, 5% Octyl | 30 |
| Breathable Sunblock | Salicylate, 3% Oxybenzone, 2% | |
| | Avobenzone, 7% Homosalate | |
| Banana Boat ®, Kids, Quik | 7.5% Octyl Methoxycinnamate, 5% | 35 |
| Blok, Spray Lotion | Octyl Salicylate, 6% Oxybenzone, | |
| | 2.5% Octocrylene, 3% Avobenzone | |
| Fruit of the Earth ®-Block | 7.5% Octyl Methoxycinnamate, 5% | 30 |
| Up with Aloe Vera | Octyl Salicylate, 5% Oxybenzone, | |
| | 2% Avobenzone, | |

TABLE II
SUNSCREEN COMPOSITION OF THE PRESENT INVENTION

| | | |
|---|---|---|
| Ingredient | Tradename/Description/Function | Weight % |

Water Phase

| | | |
|---|---|---|
| Water | | 68.970 |
| Propylene Glycol | Moisturizer | 1.000 |
| Acrylates/C10-30 alkyl acrylates | Pemulen ® TR2, Emulsifier- | 0.220 |
| crosspolymer | thickener (Noveon) | |
| Sodium Polyacrylate | Sokalan ® PA 30 CL (45% active), | 0.470 |
| | Low molecular weight weak acid | |
| | polymer (BASF) | |
| Phenoxyethanol, Methylparaben, | Phenonip ®, Preservative mixture | 1.000 |
| Butylparaben, Ethylparaben, | (Clariant) | |
| Propylparaben | | |
| Sodium Hydroxide (50% active) | pH adjustment reagent | 0.200 |
| Aloe Extract (50% active) | Skin feel enhancer | 0.150 |
| Water, Sodium bentonite (26.7% of | Polargel ® UV 1116, SPF booster | 3.750 |
| the dispersion), Titanium Dioxide | (AMCOL) | |
| (20% of the dispersion), | | |
| Lignosulfonate (5.75% of the | | |
| dispersion) | | |
| Tetrasodium ethylene diamine | Chelating agent | 0.050 |
| tetracetate | | |
| Phenoxyethanol, Chlorphenesin, | Germazide ® MPB, Preservative | 1.330 |
| Glycerin, Methylparaben, Benzoic | (Englehard) | |
| Acid | | |

Oil Phase

| | | |
|---|---|---|
| Octyl Methoxycinnamate | Sunscreen active | 5.000 |
| Octyl Salicylate | Sunscreen active | 3.000 |
| Oxybenzone | Sunscreen active | 3.000 |
| Avobenzone | Sunscreen active | 2.000 |
| Isopropyl Myristate | Emollient | 7.000 |
| Cetearyl Polyglucoside | Tego Care ® CG 90, Polymeric | 0.250 |
| | emulsifier (Degussa) | |
| Tocopheryl Acetate | Vitamin E | 0.100 |
| Retinyl Palmitate | Vitamin A | 0.010 |
| Polyethylene | Polyethylene AC 1700, Water | 2.500 |
| | resistance promoter (Honeywell) | |

Manufacturing Procedure
Heat the water phase to 85-90° C., under mixing in a rotor-stator homogenizer (Silverson laboratory homogenizer).
Heat the oil phase to 85-90° C. under gentle agitation.
Add slowly the heated oil phase to the heated water phase, while the batch remains under homogenizer mixing at 5,000-10,000 rpm of homogenizer speed.
Continue homogenizing until the batch appears uniformly mixed.
   Sun Protection Factor (SPF)

The SPF-value, 32, for the above sunscreen composition was determined based on 20-subject in-vivo SPF testing as per the very water resistant SPF testing protocol mandated by the US Federal Drug Administration.

Stability Properties

The sunscreen formulation did not show visible separation of any component, when subjected to stability tests such as the following:

i) heating the emulsion to 60° C., followed by centrifuging the heated emulsion at 3,000 rpm for 30 minutes

ii) storing the emulsion in a 45° C.-oven for 3 months

The levels of sunscreen actives contained in the sunscreen emulsion did not vary much between before and after the emulsion was placed in a 45° C.-oven, indicating good stability of the sunscreen actives during storage at 45° C.

Sunscreen Wipes

Sunscreen wipes manufactured using the sunscreen emulsion of Table II were shown to provide adequate protection of the skin from UV damage when used by panelists during outdoor activities lasting from about 2 to about 8 hours under direct sunlight in summer months. The basesheet for these sunscreen wipes comprised a non-woven fabric made from a spunlace, rayon-polyester blend material. The loading level of the sunscreen emulsion was about 400%, based on the weight of wipe basesheet.

### EXAMPLE II

This example demonstrates that the viscosity of an O-W sunscreen emulsion having a crosslinked polyacrylate used as a thickener for the water-phase of the emulsion, would be considerably higher if the emulsion further contained an SPF booster such as Polargel® UV 1116 (Table III). It further demonstrates that the sunscreen emulsion with Polargel® UV 1116 contained therein, would exhibit an increased level of shear-thinning rheology (flow-property), if it contained a low molecular weight sodium polyacrylate (Sokalan® PA 30CL, Table II) in the water phase of the emulsion.

A series of sunscreen emulsions were produced as per the formulations presented in Table III. The method used in manufacturing these emulsions is nearly similar to the procedure described in EXAMPLE I, except that Polargel® UV 1116 was added to certain emulsion batches as a post-emulsification-addition ingredient after cooling the emulsions to room temperature. Emulsion 1 did not contain either Polargel® UV 1116 or Sokalan® PA 30CL. Emulsion 2 contained 3.75% by weight of Polargel® UV 1116, but no Sokalan® PA 30CL. Emulsions 3, 4, and 5 contained the same amount of Polargel® UV 1116 as in Emulsion 2, and varying amounts of Sokalan® PA 30CL. For these emulsions, Polargel® UV 1116 and Sokalan® PA 30CL were mixed together in a Silverson-homogenizer operated at a speed of 6,000-8,000 rpm, prior to addition to an emulsion batch. In mixing Polargel® UV 1116 and Sokalan® PA 30CL, the former ingredient was first diluted with deionized water in 1:1 proportion under vigorous agitation. Sokalan® PA 30CL was subsequently added to the diluted batch of Polargel® UV 1116 for mixing in a Silverson homogenizer.

The room-temperature viscosities of these emulsions were measured at various shear rates, using a Brookfield RVT viscometer, wherein the speed of the viscometer spindle was varied in order to vary the shear rate. For each emulsion, the viscosity measurement was carried out after the emulsion had been stored at room temperature for a period of at least 16 hours after it was manufactured. Spindle 7 was used for all viscosity measurements. In measuring the viscosity, first, the spindle was inserted gently into an emulsion sample with minimal disturbance of the emulsion, after which a period of 1 minute was allowed to pass before initiating the viscometer run. Subsequently, the viscometer was turned on and the spindle was allowed to rotate for a given length of time, depending on the speed of spindle rotation, prior to recording the viscosity reading. The spindle rotation time allowed for the different spindle speeds are as follows: 2 minutes for 0.5 rpm, 1 minute for 1 rpm, 30 seconds for 2.5 rpm, and 15 seconds for each of 5 rpm, 10 rpm, and 20 rpm. The results of these viscosity measurements are given in Table IV. In Table IV, the column-label “shear-thinning index” denotes the ratio of 0.5 rpm to 20 rpm viscosities, signifying the level of shear-thinning—the higher the ratio, the greater the level of shear-thinning.

TABLE III

| | | | | | |
|---|---|---|---|---|---|
| Ingredients | Emulsion 1 | Emulsion 2 | Emulsion 3 | Emulsion 4 | Emulsion 5 |

Water Phase

| | | | | | |
|---|---|---|---|---|---|
| Water | 73.180 | 69.430 | 69.150 | 68.990 | 68.700 |
| Propylene Glycol | 1.000 | 1.000 | 1.000 | 1.000 | 1.000 |
| Pemulen ® TR2 | 0.230 | 0.230 | 0.230 | 0.230 | 0.230 |
| Tego Care ® CG 90 | 0.250 | 0.250 | 0.250 | 0.250 | 0.250 |
| Phenonip ® | 1.000 | 1.000 | 1.000 | 1.000 | 1.000 |
| 50% Sodium Hydroxide | 0.200 | 0.200 | 0.200 | 0.200 | 0.200 |
| 50% Aloe Extract | 0.150 | 0.150 | 0.150 | 0.150 | 0.150 |
| Tetrasodium ethylene | 0.050 | 0.050 | 0.050 | 0.050 | 0.050 |
| diamine tetracetate | | | | | |
| Germazide ® MPB | 1.330 | 1.330 | 1.330 | 1.330 | 1.330 |

Oil Phase

| | | | | | |
|---|---|---|---|---|---|
| Octyl | 5.000 | 5.000 | 5.000 | 5.000 | 5.000 |
| Methoxycinnamate | | | | | |
| Octyl Salicylate | 3.000 | 3.000 | 3.000 | 3.000 | 3.000 |
| Oxybenzone | 3.000 | 3.000 | 3.000 | 3.000 | 3.000 |
| Avobenzone | 2.000 | 2.000 | 2.000 | 2.000 | 2.000 |
| Isopropyl Myristate | 7.000 | 7.000 | 7.000 | 7.000 | 7.000 |
| Tocopheryl Acetate | 0.100 | 0.100 | 0.100 | 0.100 | 0.100 |
| Retinyl Palmitate | 0.010 | 0.010 | 0.010 | 0.010 | 0.010 |
| Polyethylene | 2.500 | 2.500 | 2.500 | 2.500 | 2.500 |

Post-Add Phase

| | | | | | |
|---|---|---|---|---|---|
| Polargel UV 1116 | 0 | 3.75 | 3.75 | 3.75 | 3.75 |
| Sokalan ® PA 30 CL | 0 | 0 | 0.28 | 0.44 | 0.73 |

TABLE IV

| | | | | | | | Shear- |
|---|---|---|---|---|---|---|---|
| | Brookfield Viscosity, cps | | | | | | thinning |
| Emulsion # | 0.5 rpm | 1 rpm | 2.5 rpm | 5 rpm | 10 rpm | 20 rpm | Index |
| 1 | 90,000 | 68,000 | 44,400 | 25,800 | 12,200 | 6,700 | 13.43 |
| 2 | 200,000 | 128,000 | 74,400 | 48,600 | 28,400 | 15,100 | 13.25 |
| 3 | 160,000 | 91,000 | 51,600 | 32,600 | 19,000 | 9,100 | 17.58 |
| 4 | 124,000 | 68,000 | 40,000 | 25,800 | 15,300 | 7,400 | 16.76 |
| 5 | 83,200 | 52,000 | 30,800 | 19,000 | 10,700 | 5,750 | 14.47 |

The effects of sodium polyacrylate on the rheological properties of the sunscreen compositions described herein are demonstrated further through the formulations presented in Table V, which contain a crosslinked polyacrylate-based thickening agent, Ultrez® 21 (Noveon). The Brookfield viscosities of these formulations at various spindle speeds, as measured using spindle 7 at 20-25° C., are given in Table VI.

TABLE V

| | | |
|---|---|---|
| Ingredients | Emulsion 1 | Emulsion 2 |

Water Phase

| | | |
|---|---|---|
| Water | 68.250 | 67.500 |
| Glycerin | 1.000 | 1.000 |
| Ultrez ® 21 | 0.200 | 0.200 |
| Phenonip | 1.000 | 1.000 |
| 50% Sodium Hydroxide | 0.200 | 0.200 |
| Tetrasodium ethylene | 0.100 | 0.100 |
| diamine tetracetate | | |

Oil Phase

| | | |
|---|---|---|
| Octocrylene | 5.000 | 5.000 |
| Octyl Salicylate | 5.000 | 5.000 |
| Oxybenzone | 2.000 | 2.000 |
| Avobenzone | 3.000 | 3.000 |
| Homosalate | 6.000 | 6.000 |
| Stearic Acid | 1.000 | 1.000 |
| Cetyl Phosphate | 1.000 | 1.000 |
| Polyethylene | 2.500 | 2.500 |

Post-Add Phase

| | | |
|---|---|---|
| Polargel ® UV 1116 | 3.750 | 3.750 |
| Sokalan ® PA 30 CL | 0.000 | 0.750 |

TABLE VI

| | | | | | | | Shear- |
|---|---|---|---|---|---|---|---|
| | Brookfield Viscosity, cps | | | | | | thinning |
| Emulsion # | 0.5 rpm | 1 rpm | 2.5 rpm | 5 rpm | 10 rpm | 20 rpm | Index |
| 1 | 96,000 | 48,000 | 32,000 | 21,600 | 16,000 | 11,000 | 8.73 |
| 2 | 80,000 | 34,000 | 24,000 | 16,000 | 11,600 | 7,800 | 10.26 |

### EXAMPLE III

This example demonstrates that a sunscreen composition, containing a SPF booster such as Polargel® UV 1116, may not be stable against the separation of the particulate components of the SPF booster, if the low-shear-rate viscosities of the composition, as measured on a Brookfield RVT viscometer using spindle 7 at 25° C., are not ≧50,000 cps and 30,000 cps, respectively, at 0.5 rpm and 1 rpm of spindle speeds, wherein the viscosity is measured after at least about 24 hours of room-temperature storage from the time of manufacturing of the composition.

Table VII presents sunscreen emulsion formulations that are identical, except for the dosages of the thickening agent, Pemulene® TR2, and its neutralizing agent, sodium hydroxide. The respective Brookfield viscosities of the emulsions at various spindle speeds, as measured using spindle # 7 at 25° C., are also shown in Table V. Following overnight storage at room temperature post manufacturing, emulsion 1 showed separation of the particulate components of Polargel® UV 1116, when centrifuged at 3,000 rpm for 30 minutes, after being heated to 60° C., albeit there was no separation of the oil phase. This would suggest that while a stable emulsion could be made using the emulsion 1 formulation presented in Table V, the emulsion, however, would not allow an effective use of Polargel® UV 1116 and therefore would not perform well as a high-efficiency sunscreen emulsion. On the other hand, emulsion 2 did not show any separation of the particulate components of Polargel® UV 1116, when centrifuged at 3,000 rpm for 30 minutes, after being heated to 60° C. Viscosity measurements of the sunscreen compositions were carried out following the procedure summarized in EXAMPLE II.

TABLE VII

| | | |
|---|---|---|
| Ingredients | Emulsion 1 | Emulsion 2 |

Water Phase

| | | |
|---|---|---|
| Water | 66.950 | 66.845 |
| Propylene Glycol | 1.000 | 1.000 |
| Pemulen ® TR2 | 0.100 | 0.145 |
| Phenonip ® | 1.000 | 1.000 |
| 50% Sodium Hydroxide | 0.350 | 0.410 |
| Tetrasodium ethylene | 0.100 | 0.100 |
| diamine tetracetate | | |

Oil Phase

| | | |
|---|---|---|
| Octyl Methoxy Cinnamate | 7.500 | 7.500 |
| Octyl Salicylate | 5.000 | 5.000 |
| Oxybenzone | 4.000 | 4.000 |
| Avobenzone | 2.000 | 2.000 |
| Isopropyl Myristate | 3.000 | 3.000 |
| Stearic Acid | 1.000 | 1.000 |
| Cetyl Phosphate | 1.000 | 1.000 |
| Polyethylene | 2.500 | 2.500 |

Post-Add Phase

| | | |
|---|---|---|
| Polargel ® UV 1116 | 3.750 | 3.750 |
| Sokalan ® PA 30 CL | 0.75 | 0.750 |
| Brookfield RVT Viscosity, | | |
| cps | | |
| 0.5 rpm | 32,000 | 96,000 |
| 1 rpm | 20,000 | 48,000 |
| 2.5 rpm | 9,600 | 28,800 |
| 5 rpm | 5,200 | 19,200 |
| 10 rpm | 3,200 | 11,000 |
| 20 rpm | 2,200 | 6,000 |

### EXAMPLE IV

This example presents the composition for a sunscreen emulsion of the present invention, which passed the 3-cycle freeze-thaw test noted in a previous section. The emulsion remained intact, showing no visible separation of any materials, after it was subjected to the freeze-thaw test (with −10° C. as the freezing temperature). The SPF of the emulsion was 75.8 and the very water resistant SPF was 60, wherein the SPF values were determined based on 3-subject, in-vivo SPF testing in accordance with the US FDA SPF testing protocols. Sunscreen wipes made with the foregoing emulsion showed good freeze-thaw stability.

TABLE VIII
SUNSCREEN COMPOSITION OF THE PRESENT INVENTION

| | | |
|---|---|---|
| Ingredient | Tradename/Description/Function | Weight % |

Water Phase

| | | |
|---|---|---|
| Water | | 47.318 |
| Propylene Glycol | Humectant | 5.000 |
| Urea | Humectant | 6.000 |
| Acrylates/C10-30 alkyl | Pemulen ® TR2, Emulsifier- | 0.175 |
| acrylates crosspolymer | thickener (Noveon) | |
| Sodium Polyacrylate | Sokalan ® PA 30 CL | 0.320 |
| | (45% active), Low molecular | |
| | weight weak acid polymer | |
| | (BASF) | |
| Phenoxyethanol, | Euxyl ® PE 9010, Preservative | 1.000 |
| Ethylhexylglycerin | mixture | |
| | (Schulke & Mayr) | |
| Sodium Hydroxide (50% | pH adjustment reagent | 0.127 |
| active) | | |
| Aloe Extract (50% | Skin feel enhancer | 0.150 |
| active) | | |
| Water, Sodium bentonite, | Polargel ® UV, SPF booster | 8.000 |
| Titanium Dioxide, | (AMCOL) | (0.373% |
| Kaolin, Lignosulfonate, | | Sodium |
| Sodium Polyacrylate | | Polyacrylate |
| (Sokalan ® PA 30 CL) | | Solution |

Oil Phase

| | | |
|---|---|---|
| Octyl Salicylate | Sunscreen active | 5.000 |
| Oxybenzone | Sunscreen active | 6.000 |
| Avobenzone | Sunscreen active | 3.000 |
| Homosalate | Sunscreen Active | 15.000 |
| Methyl Glucose | Tego Care ® PS, Emulsifier | 0.800 |
| Sesquistearate | (Degussa) | |
| Tocopheryl Acetate | Vitamin E | 0.100 |
| Retinyl Palmitate | Vitamin A | 0.010 |
| Vinylpyrrolidone/ | Ganex V-220F, Water resistance | 2.000 |
| Eicosene Copolymer | promoter (International Specialty | |
| | Products) | |

Manufracturing Procedure
Combine the oil phase and heat the mixture to 60-65° C., under agitation until the solids dissolve completely.
Cool the heated oil phase to the ambient temperature.
Combine the water phase ingredients under mixing in a rotor-stator homogenizer (Silverson laboratory homogenizer). Continue mixing until the composition looks uniform or lump-free.
Add slowly the oil phase to the water phase, while the batch remains under homogenizer mixing at 5,000-10,000 rpm of homogenizer speed.
Continue homogenizing until the batch appears uniformly mixed.

The Brookfield viscosities of the above emulsion, measured at various spindle speeds, using spindle # 7 are presented in Table IX.

TABLE IX
Brookfield Viscosity

| Spindle Speed, | |
|---|---|
| Rpm | Viscosity, CPS |
| 0.5 | 112,000 |
| 1 | 60,000 |
| 2.5 | 33,600 |
| 5 | 18,400 |
| 10 | 8,400 |
| 20 | 4,800 |

What is claimed is:

1. A sunscreen wipe comprising a water-insoluble wipe substrate comprising synthetic and/or natural fibers, impregnated with a sunscreen composition containing (i) a sunscreen active, (ii) a particulate-based additive for boosting the sun protection factor (SPF) of the sunscreen composition, and (iii) an additive, soluble in the said sunscreen composition, that can enhance the shear-thinning property of the sunscreen composition, the said sunscreen composition meeting the following specifications:
a) for a dosage of 1.5% by weight of component (ii), the in-vivo SPF of the sunscreen composition is ≧1.8 times the weight percent of the sunscreen active contained therein, based on the weight of the sunscreen composition;
b) without any sunscreen active contained therein, a composition containing 1.5% by weight of component (ii) yields an SPF of <2;
c) the shear thinning index of the sunscreen composition is greater by a factor of at least 1.05 with the component (iii) contained therein than without the component (iii), the shear thinning index being defined as the ratio of viscosities of the sunscreen composition at 0.5 rpm and 20 rpm, measured on a Brookfield RVT viscometer at 25° C., using spindle 7, after cooling the composition to room temperature and storing it at that temperature for about 24 hours.

2. The sunscreen wipe of claim 1 wherein the ratio of the weight of the sunscreen composition impregnated into the wipe substrate to the weight of the wipe substrate is 20:1-1:1.

3. The sunscreen wipe of claim 2 wherein the ratio of the weight of the sunscreen composition impregnated into the wipe substrate to the weight of the wipe substrate is 10:1-2:1.

4. The sunscreen wipe of claim 3 wherein the ratio of the weight of the sunscreen composition impregnated into the wipe substrate to the weight of the wipe substrate is 6:1-3:1.

5. The sunscreen wipe of claim 1 wherein the sunscreen active is an organic UV-absorbing material selected from the group consisting of octyl methoxycinamate, homosalate, octocrylene, octyl salate, methylbenzylidene camphor, phenylbenzimidazole sulfonic acid, ethylhexyl triazone, oxybenzone, methyl anthranilate, avobenzone, 3-benzylidene camphor, 4-methylbenzylidene camphor, benzylidene camphor sulfonic acid, camphor benzalkonium methosulfate, isoamyl-p-methoxycinnamate, isopropylbenzyl salicylate, and mixtures thereof.

6. The sunscreen wipe of claim 5 wherein the amount of the sunscreen active is no less than 2%, based on the weight of the sunscreen composition impregnating the wipe.

7. The sunscreen wipe of claim 1 wherein the particulate-based SPF booster comprises 0.1-25% by weight of the sunscreen composition impregnating the wipe.

8. The sunscreen wipe of claim 7 wherein the particulate-based SPF booster comprises 0.25-10% by weight of the sunscreen composition impregnating the wipe.

9. The sunscreen wipe of claim 8 wherein the particulate-based SPF booster comprises 0.5-2% by weight of the sunscreen composition impregnating the wipe.

10. The sunscreen wipe of claim 1 wherein the shear-thinning index-boosting additive comprises 0.025-5% by weight of the sunscreen composition impregnating the wipe.

11. The sunscreen wipe of claim 10 wherein the shear-thinning index-boosting additive comprises 0.1-2% by weight of the sunscreen composition impregnating the wipe.

12. The sunscreen wipe of claim 11 wherein the shear-thinning index-boosting additive comprises 0.5-1% by weight of the sunscreen composition impregnating the wipe.

13. A method of protecting human skin from UV radiation comprising of contacting the sunscreen wipe of claim 1 against the skin, thereby transferring the sunscreen composition from the wipe substrate to the skin.

14. A sunscreen composition, either impregnating a wipe substrate or comprising a sunscreen spray, wherein the composition comprises an oil phase dispersed stably as emulsion droplets in a water phase that contains i) a booster for the sun protection factor (SPF), comprising a combination of water-dispersible particulate materials, and a water-soluble or water-dispersible phenolic polymer; and ii) a water-soluble or water-dispersible polymer having a weak acid group, a weight average molecular weight of 1,000-100,000 Dalton, and an anionic charge density of no less than 1.5 milliequivalent per gram of the polymer; the said sunscreen composition meeting the following specifications:
(a) the in-vivo sun protection factor (SPF) is ≧1.8 times the weight percent of the sunscreen active(s) contained therein, based on the weight of the sunscreen composition; and
(b) the shear thinning index of the composition is greater by a factor of at least 1.05 with the weak acid polymer contained therein than without the polymer, the shear thinning index being defined as the ratio of viscosities of the sunscreen composition at 0.5 rpm and 20 rpm, measured on a Brookfield RVT viscometer at 25° C., using spindle 7, after cooling the composition to room temperature and storing it at that temperature for about 24 hours.

15. The sunscreen composition of claim 14 wherein the low-shear-rate viscosities are at least 50,000 cps at 0.5 rpm and 35,000 cps at 1 rpm, while the high-shear-rate viscosities are 10,000-30,000 cps at 5 rpm and 2,500-10,000 cps at 20 rpm, as measured on a Brookfield RVT viscometer, at 25° C., using spindle # 7, after cooling the composition to room temperature and storing it at that temperature for about 24 hours.

16. The sunscreen composition of claim 14 wherein the emulsion does not show any separation of either the oil phase or the particulate components of the water phase, when centrifuged at 3,000 rpm for 30 minutes after being heated to 60° C.

17. The sunscreen composition of claim 14 wherein the emulsion does not show any visible separation of either the oil phase or the particulate components of the water phase, after three freeze-thaw cycles, wherein during each cycle, the sunscreen composition is stored in a freezer at −10° C. for a period of 24 hours, followed by thawing the composition to room temperature.

18. The sunscreen composition of claim 14 wherein oil phase contains one or more organic UV-absorbing sunscreen actives selected from the group consisting of octyl methoxycinamate, homosalate, octocrylene, octyl salate, methylbenzylidene camphor, phenylbenzimidazole sulfonic acid, ethylhexyl triazone, oxybenzone, methyl anthranilate, avobenzone, 3-benzylidene camphor, 4-methylbenzylidene camphor, benzylidene camphor sulfonic acid, camphor benzalkonium methosulfate, isoamyl-p-methoxycinnamate, isopropylbenzyl salicylate, and mixtures thereof.

19. The sunscreen composition of claim 18 wherein the amount of the organic sunscreen active is no less than 2%, based on the weight of the composition.

20. The sunscreen composition of claim 14 wherein the particulate materials of the SPF booster are selected from the group consisting of inorganic oxides, water-insoluble inorganic salts, silicate minerals, smectite clays, and latex polymers.

21. The sunscreen composition of claim 20 wherein at least one of the particulate materials has an average primary particle size of less than 1 micron.

22. The sunscreen composition of claim 14 wherein the particulate materials of the SPF booster, comprise 0.1-25% by weight of the composition.

23. The sunscreen composition of claim 22 wherein the particulate materials of the SPF booster, comprise 0.25-10% by weight of the composition.

24. The sunscreen composition of claim 23 wherein the particulate materials of the SPF booster, comprise 0.5-2% by weight of the composition.

25. The sunscreen composition of claim 14 wherein the phenolic polymer is selected from the group consisting of lignosulfonate, lignin, oxylignin, humate, and mixtures thereof.

26. The sunscreen composition of claim 25 wherein the phenolic polymer comprises 0.05-2% by weight of the composition.

27. The sunscreen composition of claim 26 wherein the phenolic polymer comprises 0.1-1% by weight of the composition.

28. The sunscreen composition of claim 27 wherein the phenolic polymer comprises 0.2-0.5% by weight of the composition.

29. The sunscreen composition of claim 14 wherein the weak acid group is selected from the group consisting of carboxylate, phosphate, phosphonate, phenolate, and mixtures thereof.

30. The composition of claim 29 wherein the weak acid polymer is sodium polyacrylate.

31. The sunscreen composition of claim 29 wherein the weak acid polymer comprises 0.025-5% by weight of the composition.

32. The sunscreen composition of claim 31 wherein the weak acid polymer comprises 0.1-2% by weight of the composition.

33. The sunscreen composition of claim 32 wherein the weak acid polymer comprises 0.5-1% by weight of the composition.

34. A sunscreen wipe comprising:
a water-insoluble wipe substrate comprising synthetic and/or natural fibers impregnated with the sunscreen composition of claim 14.

35. The sunscreen wipe of claim 34 wherein the ratio of the weight of the sunscreen composition impregnated into the wipe substrate to the weight of the wipe substrate is 20:1-1:1.

36. The sunscreen wipe of claim 35 wherein the ratio of the weight of the sunscreen composition impregnated into the wipe substrate to the weight of the wipe substrate is 10:1-2:1.

37. The sunscreen wipe of claim 36 wherein the ratio of the weight of the sunscreen composition impregnated into the wipe substrate to the weight of the wipe substrate is 6:1-3:1.

38. A method of protecting human skin from UV radiation comprising of contacting the sunscreen wipe of claim 34 against the skin, thereby transferring the sunscreen composition from the wipe substrate to the skin.

39. A sunscreen composition, either impregnating a wipe substrate or comprising a sunscreen spray, wherein the composition comprises an oil phase containing a UV-absorbing organic sunscreen active, said oil phase dispersed in a stable emulsion in a water phase that contains i) a booster for the sun protection factor (SPF), comprising a combination of water-dispersible particulate materials, and a water-soluble or water-dispersible phenolic polymer; and ii) a water-soluble or water-dispersible polymer having a weak acid group, a weight average molecular weight of 1,000-100,000 Dalton, and an anionic charge density of no less than 1.5 milliequivalents per gram of the polymer.

40. The sunscreen composition of claim 39, wherein the organic sunscreen active is selected from the group consisting of octyl methoxycinamate, homosalate, octocrylene, octyl salate, methylbenzylidene camphor, phenylbenzimidazole sulfonic acid, ethylhexyl triazone, oxybenzone, methyl anthranilate, avobenzone, 3-benzylidene camphor, 4-methylbenzylidene camphor, benzylidene camphor sulfonic acid, camphor benzalkonium methosulfate, isoamyl-p-methoxycinnamate, isopropylbenzyl salicylate, and mixtures thereof.

41. The sunscreen composition of claim 39, wherein the amount of the organic sunscreen active is no less than 2%, based on the weight of the composition.

42. The sunscreen composition of claim 39, wherein one of the particulate materials of the SPF booster is smectite clay and the other particulate material is selected from the group consisting of inorganic oxides, water-insoluble inorganic salts, silicate minerals, and latex polymers.

43. The sunscreen composition of claim 42, wherein at least one of the particulate materials has an average primary particle size of less than 1 micron.

44. The sunscreen composition of claim 26, wherein the particulate materials of the SPF booster comprise 0.1-25% by weight of the composition.

45. The sunscreen composition of claim 31, wherein the particulate materials of the SPF booster comprise 0.25-10% by weight of the composition.

46. The sunscreen composition of claim 32, wherein the particulate materials of the SPF booster comprise 0.5-2% by weight of the composition.

47. The sunscreen composition of claim 39, wherein the phenolic polymer is selected from the group consisting of lignosulfonate, lignin, oxylignin, humate, and mixtures thereof.

48. The sunscreen composition of claim 47, wherein the phenolic polymer comprises 0.05-2% by weight of the composition.

49. The sunscreen composition of claim 48, wherein the phenolic polymer comprises 0.1-1% by weight of the composition.

50. The sunscreen composition of claim 49, wherein the phenolic polymer comprises 0.2-0.5% by weight of the composition.

51. The sunscreen composition of claim 39, wherein the weak acid group is selected from the group consisting of carboxylate, phosphate, phosphonate, phenolate, and mixtures thereof.

52. The composition of claim 51, wherein the weak acid polymer is sodium polyacrylate.

53. The sunscreen composition of claim 51, wherein the weak acid polymer comprises 0.025-5% by weight of the composition.

54. A sunscreen wipe comprising:
a water-insoluble wipe substrate comprising synthetic and/or natural fibers impregnated with the sunscreen composition of claim 39.

55. The sunscreen wipe of claim 54, wherein the ratio of the weight of the sunscreen composition impregnated into the wipe substrate to the weight of the wipe substrate is 20:1-1:1.

56. A method of protecting human skin from UV radiation comprising contacting the sunscreen wipe of claim 54 against the skin, thereby transferring the sunscreen composition from the wipe substrate to the skin.
